(19) United States
(12) Patent Application Publication
(10) Pub. No.: US 2026/0158297 A1
(43) Pub. Date: Jun. 11, 2026

(54) ULTRASOUND IMAGE-GUIDED FOCUSED ULTRASOUND SYSTEMS AND RELATED METHODS

(71) Applicant: RESONANT ACOUSTICS INTERNATIONAL INC., Markham (CA)

(72) Inventors: Nicholas Chris CHAGGARES, Whitby (CA); Varak KALIAN, North York (CA); Khachic KARSHAFIAN, North York (CA); Eric RIEDER, Georgetown (CA)

(73) Assignee: RESONANT ACOUSTICS INTERNATIONAL INC., Markham, ON (CA)

(21) Appl. No.: 19/127,168

(22) PCT Filed: Nov. 14, 2023
(86) PCT No.: PCT/CA2023/051514

§ 371 Date: May 5, 2025

Related U.S. Application Data
(60) Provisional application No. 63/383,676, filed on Nov. 14, 2022.

(51) Int. Cl. A61N 7/02; A61N 7/00; B06B 1/06

(52) U.S. Cl. CPC A61N 7/02; B06B 1/0622; A61N 2007/0078

(57) ABSTRACT

There is provided a transducer including an acoustic stack including a composite piezoelectric layer, the composite piezoelectric comprising a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture. The acoustic stack also includes a thermally and electrically conductive layer, a thermally conductive and electrically isolating layer, a lens, and at least one matching layer. The acoustic stack is configured to produce a field defining an acoustic focal zone, the acoustic focal zone having a wide lateral dimension, a narrow lateral dimension, and a low focal length to focal width ratio. The transducer also includes a backing structure contacting the rear end of the acoustic stack.

### TECHNICAL FIELD

The technical field generally relates to the field of acoustic energy and more particularly relates to ultrasound image-guided focused ultrasound systems and related methods.

### BACKGROUND

There is a need for single element focused ultrasound transducers (referred to as “FUS”) that can produce wide acoustic focal zones that are relatively short in focal length without the need to change frequencies or limit treatment power, as many FUS treatment methods require certain acoustic frequencies and other parameters to achieve required results. As anatomy size and position below the skin varies, it becomes necessary to have differing focal zones (FZ) to effectively treat different anatomical structures, or portion(s) thereof, while minimizing the risk of damaging skin either thermally or otherwise.

There is still a need for techniques, apparatus, devices, and methods that alleviate or mitigate the problems of prior art.

### SUMMARY

The present techniques generally concern a relatively high aspect ratio aperture focused ultrasound (HARFUS) transducer with thermally efficient electrically isolated acoustically matched lens. The present techniques also relate to a modular high aspect ratio single element focused ultrasound transducer. In some embodiments, the transducer enables one shot treatment of wide and even square aspect ratio focal zones, which can allow for transcutaneous single sonication treatments or a round, superficial nerve having a large diameter compared to the ideal FUS wavelength. In some embodiments, the transducer is configured for generating an acoustic focal zone having a pumpkin seed shape, i.e., wide and short in the lateral (to for example a nerve) treatment plane, and relatively thin in the orthogonal direction. In some embodiments, the shape and dimensions of the focal zone can be tailored to allow neuromodulation over the entire cross section of nerves in one sonication. In accordance with one aspect, there is provided a high aspect ratio aperture focused ultrasound (HARFUS) transducer, the HARFUS transducer including a high aspect ratio acoustic stack having a front end and a rear end, and a backing structure contacting the rear end of the acoustic stack.

The acoustic stack includes: a composite piezoelectric layer, the composite piezoelectric including a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture; a thermally and electrically conductive layer in physical contact with the piezoelectric layer, the composite piezoelectric layer being acoustically matched with the thermally and electrically conductive layer; a thermally conductive and electrically isolating layer in contact with the thermally and electrically conductive layer; a lens in contact with the thermally conductive and electrically isolating layer; and at least one matching layer at least partially extending over the lens, wherein the acoustic stack is configured to produce a field defining an acoustic focal zone, the acoustic focal zone having a relatively wide lateral dimension, a relatively narrow lateral dimension, and a relatively low focal length to focal width ratio.

In some embodiments, the acoustic focal zone is shaped like a pumpkin seed. In geometric terms, the acoustic focal zone may have an axial length to lateral width ratio of less than 3:1 for example. In some embodiments, the acoustic focal zone may also have a second orthogonal lateral width of less than ½ of the first lateral width.

In some embodiments, said at least one matching layer includes a front matching layer extending over a rear matching layer.

In some embodiments, the lens has tapered edges and a tapered width.

In some embodiments, the lens has an outer periphery, the outer periphery being threaded or including anechoic features.

In some embodiments, the lens curvature is a spherical curvature or an elliptical curvature.

In some embodiments, the HARFUS transducer further includes a de-matching layer contacting the rear end of the high aspect ratio acoustic stack, the de-matching layer directly contacting one of: the composite piezoelectric layer and the backing structure.

In some embodiments, the backing structure is a dual layer de-matching backing structure.

In some embodiments, the HARFUS transducer further includes a heat sink.

In accordance with one aspect, there is provided a modular system, the modular system including a plurality of HARFUS transducers as disclosed herein, arranged to result in a laterally merged focal zone having a lower axial focal length to lateral focal width ratio than a single HARFUS transducer.

In accordance with one aspect, there is provided a high aspect ratio aperture focused ultrasound (HARFUS) transducer, the HARFUS transducer including a high aspect ratio acoustic stack having a front end and a rear end, and a backing structure contacting the rear end of the high aspect ratio acoustic stack. The acoustic stack includes a composite piezoelectric layer, the composite piezoelectric including a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture; a thermally and electrically conductive layer in physical contact with the piezoelectric layer, the composite piezoelectric layer being acoustically matched with the thermally and electrically conductive layer; a lens in contact with the thermally conductive and electrically isolating layer; and at least one matching layer at least partially extending over the lens, wherein the acoustic stack is configured to produce a field defining an acoustic focal zone, the acoustic focal zone having a relatively wide lateral dimension, a relatively narrow lateral dimension, and a relatively low focal length to focal width ratio.

In some embodiments, the acoustic focal zone is shaped like a pumpkin seed.

In some embodiments, said at least one matching layer includes a front matching layer extending over a rear matching layer.

In some embodiments, the lens has tapered edges and a tapered width.

In some embodiments, the lens has an outer periphery, the outer periphery including anechoic features such as wedges or threads or an acoustically absorbing material.

In some embodiments, the lens curvature is a spherical curvature or an elliptical curvature.

In some embodiments, the HARFUS transducer further includes a de-matching layer contacting the rear end of the high aspect ratio acoustic stack, the de-matching layer directly contacting one of: the composite piezoelectric layer and the backing structure.

In some embodiments, the backing structure is a dual layer de-matching backing structure.

In some embodiments, the HARFUS transducer further includes a heat sink.

A modular system, the modular system including a plurality of HARFUS transducers as disclosed herein, arranged to result in a laterally merged focal zone having a lower axial focal length to lateral focal width ratio than a single HARFUS transducer.

In accordance with one aspect, there is provided a method for aligning a treatment head including a HARFUS transducer and a diagnostic imaging system, with a sample to characterize, the method including acoustically coupling the treatment head with an ultrasound-sensitive phantom; performing a co-registration sonication sequence with the HARFUS transducer to determine an acoustic focal zone using the ultrasound-sensitive phantom; positioning an imaging plane of the diagnostic imaging system within the treatment head in a center of the acoustic focal zone of the HARFUS transducer; locking a position of the HARFUS transducer with respect to the treatment head; acoustically coupling the treatment head with respect to the sample to characterize, based on the locked positioned of the HARFUS transducer; and operating the treatment head to characterize the sample being guided by the diagnostic imaging system within the treatment head.

In accordance with one aspect, there is provided a modular HARFUS transducer configured to produce an acoustic focal zone resembling a pumpkin seed shape, having a relatively wide lateral dimension, a relatively narrow lateral dimension, and a relatively low focal length to focal width ratio. The modular HARFUS includes a plurality of HARFUS transducers, which are arranged together as to form a module. In these embodiments, the focal zone of each modular transducer may be directed towards a common spot or zone, such that the effective −3 dB focal zone can be effectively increased in width but not depth, thereby resulting in a focal width to focal length ratio of 1:1. In some embodiments, the focal zone may be wider than the length. A focal length ratio of about 1:1 allows treating the full cross section of a nerve with almost no unintended high intensity energy existing outside of the targeted nerve or other structure. In some embodiments, the transducers can be used for neuromodulation. In these embodiments, the transducer can treat the entire nerve in one sonication. Such treatment can be facilitated by a transducer that can produce a focal zone closely tailored to the target anatomy, such as the one herein presented.

Other features and advantages of the present description will become more apparent upon reading of the following non-restrictive description of specific embodiments thereof, given by way of example only with reference to the accompanying drawings.

### BRIEF DESCRIPTION OF THE DRAWINGS

FIG. 1 is an isometric view of an exemplary embodiment a high aspect ratio focused ultrasound transducer having a 1:3 ratio aperture with a single focal length, resulting in a 1:3 ratio f-number ratio.

FIG. 2 is a cross sectional view of the HARFUS transducer from FIG. 1, illustrating aspects of the exemplary embodiment including the cooling structure as well as the acoustic stack, the electrically isolating acoustically matched ceramic composite layer, and the thermally conductive lens.

FIG. 3 is an illustration depicting the high aspect ratio focal zone and accompanying acoustic field produced by the exemplary HARFUS transducer shown in FIG. 1.

FIG. 4 is an illustration of an alternative viewpoint showing the wide lateral dimension of the HARFUS focal zone.

FIG. 5 is an illustration of the acoustic field of a 1:4.5 ratio HARFUS acoustic field overlaying a hypothetical 3.5 mm cylindrical structure, such as a nerve, being bisected by the high aspect ratio focal zone of the HARFUS transducer, the −3 dB focal zone having a width of 4 mm lateral focal zone width and ˜8 mm focal length at 1.5 MHz, occurring approximately 50 mm from the face of the HARFUS transducer.

FIG. 6 is a representation of three 1:3 F-Number HARFUS transducers co-aligned to form a single merged lateral focal zone having a first −3 dB width of ˜12 mm, a second orthogonal width of 1.4 mm, and a height of ˜18 mm.

FIG. 7 is an alternative view of the three modular co-aligned HARFUS transducers shown in FIG. 6.

FIG. 8 is an exemplary embodiment of a smaller HARFUS transducer also operating at 1.5 MHz, but appropriate for small animal neuromodulation producing a focal zone having a first−3 dB width of 2.75 mm, a second orthogonal width of 0.8 mm, and a 6.4 mm long focal zone occurring about 18 mm from the face of the transducer with F-number ratio 1:4.5 at 1.5 MHz. This embodiment illustrates the ability to partially decouple the focal zone size and location from the frequency by using both the size and high aspect ratio of the aperture to control focal zone length and width.

FIG. 9 is a cross section view of the HARFUS transducer from FIG. 8. Note that in this exemplary embodiment, the lens is not electrically isolated from the ground layer as in the exemplary embodiment of FIG. 1.

FIG. 10 is a close-up view of the acoustic stack of the transducer in FIG. 8.

FIG. 11 is a series of figures illustrating the size and location of the −3 dB focal zone of the exemplary 1.5 MHz HARFUS transducer shown in FIG. 1.

FIG. 12 is a series of figures illustrating the size and location of the −3 dB focal zone of the exemplary 1.5 MHz HARFUS transducer shown in FIG. 1.

### DETAILED DESCRIPTION

In the following description, similar features in the drawings have been given similar reference numerals, and, to not unduly encumber the figures, some elements may not be indicated on some figures if they were already identified in one or more preceding figures. It should also be understood herein that the elements of the drawings are not necessarily depicted to scale, since emphasis is placed upon clearly illustrating the elements and structures of the present embodiments. The terms “a”, “an” and “one” are defined herein to mean “at least one”, that is, these terms do not exclude a plural number of elements, unless stated otherwise. It should also be noted that terms such as “substantially”, “generally” and “about”, that modify a value, condition or characteristic of a feature of an exemplary embodiment, should be understood to mean that the value, condition or characteristic is defined within tolerances that are acceptable for the proper operation of this exemplary embodiment for its intended application.

In the present description, the terms “connected”, “coupled”, and variants and derivatives thereof, refer to any connection or coupling, either direct or indirect, between two or more elements. The connection or coupling between the elements may be acoustical, mechanical, physical, optical, operational, electrical, wireless, or a combination thereof.

The terms “match”, “matching” and “matched” are intended to refer herein to a condition in which two elements are either the same or within some predetermined tolerance of each other. That is, these terms are meant to encompass not only “exactly” or “identically” matching the two elements but also “substantially”, “approximately” or “subjectively” matching the two elements, as well as providing a higher or best match among a plurality of matching possibilities.

In the present description, the expression “based on” is intended to mean “based at least partly on”, that is, this expression can mean “based solely on” or “based partially on”, and so should not be interpreted in a limited manner. More particularly, the expression “based on” could also be understood as meaning “depending on”, “representative of”, “indicative of”, “associated with” or similar expressions.

It will be appreciated that positional descriptors indicating the position or orientation of one element with respect to another element are used herein for ease and clarity of description and should, unless otherwise indicated, be taken in the context of the figures and should not be considered limiting. It will be understood that spatially relative terms (e.g., “outer” and “inner”, “outside” and “inside”, “periphery” and “central”, “over” and “under”, and “top” and “bottom”) are intended to encompass different positions and orientations in use or operation of the present embodiments, in addition to the positions and orientations exemplified in the figures.

In the context of the current disclosure, the following reference numbers may be used:
1—Front matching layer.
2—Rear matching layer.
3—Lens. Thermally efficient aluminum or other material lens, width and edges tapered to allow for adjacent assembly into a multi module treatment head.
4—Acoustically matched thermally conductive composite electrically isolating layer.
5—Thermally and electrically conductive layer—also acts as redundant ground.
6—Hi aspect ratio rectangular aperture material specific single element piezo composite matched to aluminum ˜17 MR. Can be elliptical aperture or other high aspect ratio shape to optimize acoustic field as required.
7—De-matching layer-high acoustic impedance and ideally thermally and electrically conductive such as tungsten, tungsten carbide or other molybdenum for example.
8—DLDB ¼ lambda layer, low acoustic impedance and electrically and thermally conductive such as graphite.
9—DLDB ¼ lamda layer, high acoustic impedance layer, electrically and thermally conductive such as tungsten.
10—DLDB ¼ lambda layer, low acoustic impedance and electrically and thermally conductive such as graphite.
11—DLDB layer, high acoustic impedance layer and electrically and thermally conductive, such as copper or tungsten or other conductive high acoustic impedance material.
12—Aluminum nitride or other thermally conductive but electrically insulating material.
13—A and B liquid cooling block base and liquid cooling block body for copper liquid cooling block. Could also use air based cooling solutions etc.
14—Aluminum nitride or other thermally conductive electrically insulating material, forming the thermal return path plate to couple the thermal energy from the front (5) plate to the back of the liquid cooling block.
15—Copper housing Lid, electrically and thermally conductive forming electrical shield and possible ground connection path for RF connector (not shown).
16—Liquid cooling inlet/outlet tubes.
17—Electrically and thermally conductive housing, forming both the electrical ground connection to the transducer and the thermal cooling path for the front face of the piezocomposite.
18—Thermally and electrically conductive lens forming both ground connection to the piezocomposite and the acoustic lens as well as the thermal cooling path for the front face of the piezocomposite.
19—Signal wire connected between RF connector and rear electrode of acoustic stack.
20—RF coaxial connector connected to signal wire and to ground through 17, the electrically and thermally conductive housing.
21—Anechoic features.
22—Pictorial representation of the extent of the acoustic field produced by the HARFUS transducer.
23—Pictorial representation of the −3 dB extents of acoustic field comprising the focal zone of an exemplary HARFUS transducer having a 1:3 F-Number ratio.
24—Pictorial representation of the −3 dB extents of acoustic field comprising the focal zone of an exemplary HARFUS transducer having a 1:4.5 F-Number ratio.
25—Exemplary cylindrical target, representing a typical nerve or other cylindrical FUS target.

While the embodiments of the HARFUS transducer that will be described throughout the description will be described as including a piezoelectric material, one skilled in the art would note that the HARFUS transducers of the current disclosure may instead include any ferroelectric materials, any single crystals or polycrystalline materials, any electromechanical transduction materials, such materials having one or more of the following properties: ferroelectricity, pyroelectricity, piezoelectricity, electrostriction and/other relevant properties. It will be noted that, in the context of the present description, the expression “piezoelectric material” may also refer to ferroelectric material, pyroelectric material, relaxor material and electrostrictive material, as it would be readily understood by one skilled in the art.

The description generally relates to a relatively high aspect ratio aperture focused ultrasound (HARFUS) transducer with thermally efficient electrically isolated acoustically matched lens. Embodiments of the HARFUS transducer that will be herein described include an aperture that has a relatively long axis in a first direction, and a relatively short axis in a second direction. In some embodiments, the aperture may have a length twice the size of a width. In some embodiments, the aperture may have a length three times the size of a width. In some embodiments, the ratio may be 4.5:1. The focus of the HARFUS transducer is conditioned by a lens. In some embodiments, the lens may be a spherical lens, an elliptical lens, or any other refractive focusing lens(es) which would allow conditioning the ultrasounds into a point or a focal zone. In some embodiments, the lens may have a symmetric design, which would allow producing a high aspect ratio focal zone at (or near) the focus of the lens.

In accordance with one aspect, there is provided a high aspect ratio aperture focused ultrasound (HARFUS) transducer, the HARFUS transducer including a high aspect ratio acoustic stack having a front end and a rear end, and a backing structure contacting the rear end of the acoustic stack. The acoustic stack includes: a composite piezoelectric layer, the composite piezoelectric including a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture; a thermally and electrically conductive layer in physical contact with the piezoelectric layer, the composite piezoelectric layer being acoustically matched with the thermally and electrically conductive layer; a thermally conductive and electrically isolating layer in contact with the thermally and electrically conductive layer; a lens in contact with the thermally conductive and electrically isolating layer; and at least one matching layer at least partially extending over the lens, wherein the acoustic stack is configured to produce a field defining an acoustic focal zone, the acoustic focal zone having a relatively wide lateral dimension, a relatively narrow lateral dimension, and a relatively low focal length to focal width ratio.

In some embodiments, the composite piezoelectric layer includes individual pillars, such as, for example, 1 3 or 2 2 composite piezoelectric pillars. The aperture is associated with a high aspect ratio, i.e., the whole composite piezoelectric layer is in the form of a long narrow strip or high aspect ratio ellipse for example. Of note, the composite piezoelectric layer is acoustically matched to the thermally and electrically conductive layer. In some embodiments, the thermally and electrically conductive layer could be only thermally conductive, and could be sputtered or otherwise coated with an electrically conductive layer.

In some embodiments, the acoustic focal zone is shaped like a pumpkin seed.

In some embodiments, said at least one matching layer includes a front matching layer extending over a rear matching layer.

In some embodiments, the lens has tapered edges and a tapered width.

In some embodiments, the lens has an outer periphery, the outer periphery being threaded or otherwise featuring an anechoic structure or coating.

In some embodiments, the lens curvature is a spherical curvature or an elliptical curvature.

In some embodiments, the HARFUS transducer further includes a de-matching layer contacting the rear end of the high aspect ratio acoustic stack, the de-matching layer directly contacting one of: the composite piezoelectric layer and the backing structure.

In some embodiments, the backing structure is a dual layer de-matching backing structure.

In some embodiments, the HARFUS transducer further includes a heat sink.

In accordance with one aspect, there is provided a modular system, the modular system including a plurality of HARFUS transducers as disclosed herein, arranged to result in a laterally merged focal zone having a lower axial focal length to lateral focal width ratio than a single HARFUS transducer.

In accordance with one aspect, there is provided a high aspect ratio aperture focused ultrasound (HARFUS) transducer, the HARFUS transducer including a high aspect ratio acoustic stack having a front end and a rear end, and a backing structure contacting the rear end of the high aspect ratio acoustic stack. The acoustic stack includes a composite piezoelectric layer, the composite piezoelectric including a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture; a thermally and electrically conductive layer in physical contact with the piezoelectric layer, the composite piezoelectric layer being acoustically matched with the thermally and electrically conductive layer; a lens in contact with the thermally and electrically conductive layer; and at least one matching layer at least partially extending over the lens, wherein the acoustic stack is configured to produce a field defining an acoustic focal zone, the acoustic focal zone having a relatively wide lateral dimension, a relatively narrow lateral dimension, and a relatively low focal length to focal width ratio.

In some embodiments, the acoustic focal zone is shaped like a pumpkin seed.

In some embodiments, said at least one matching layer includes a front matching layer extending over a rear matching layer.

In some embodiments, the lens has tapered edges and a tapered width.

In some embodiments, the lens has an outer periphery, the outer periphery being threaded.

In some embodiments, the lens curvature is a spherical curvature or an elliptical curvature.

In some embodiments, the HARFUS transducer further includes a de-matching layer contacting the rear end of the high aspect ratio acoustic stack, the de-matching layer directly contacting one of: the composite piezoelectric layer and the backing structure.

In some embodiments, the backing structure is a dual layer de-matching backing structure.

In some embodiments, the HARFUS transducer further includes a heat sink.

A modular system, the modular system including a plurality of HARFUS transducers as disclosed herein, arranged to result in a laterally merged focal zone having a lower axial focal length to lateral focal width ratio than a single HARFUS transducer.

In some embodiments, the system may comprise a liquid cooled FUS transducer contained in an articulating treatment head further including a micro-positioning and clamping mechanism providing for the co-registration of the imaging plane of a suitable high-resolution ultrasonic diagnostic imaging ultrasound (DIUS) with the focal zone (FZ) of the FUS transducer, or other diagnostic imaging scan head such as a wobbler-based scanner. Additionally, the system may include a water bath and a small animal handling platform with a positioning system for orienting the co-registered FUS and imaging transducers with respect to the small animal (e.g., a rat) enabling the precise visualization of peripheral nerve and subsequent insonation to achieve an accurate peripheral nerve block. In some embodiments, the liquid cooled FUS transducer may include an acoustic stack technology, allowing highly thermally efficient operation and making it possible to perform FUS treatments at up to 100% duty cycle for long durations with almost no heating of the device or lens face (or only minimal heating of the device or lens face), enabling a very wide parameter space to be tested. The FUS transducer of this system includes a high lateral aspect ratio aperture and lens technology, resulting in a relatively shorter focal length while providing the required focal width to treat the entire peripheral nerve in one sonication. To that end, one exemplary embodiment of a 1.5 MHz HARFUS transducer having a 1:4.5 f-number ratio and a focal depth of 18 mm is configured to produce a focal spot suitable for small animal PNB research, having a nominal−3 dB azimuthal focal width of approximately 2.5 mm, an elevation focal width of <about 1.0 mm and a nominal−3 dB focal length of approximately 6.5 mm, with the center of the focal spot occurring approximately 18 mm from the face of the transducer. These FZ parameters are expected to facilitate accurate treatment of the small animal peripheral nerve with one sonication while minimizing the possibility of skin burns. Co-registration (CR) of the high-resolution diagnostic imaging array to the FZ of the FUS transducer can be accomplished by positioning the DIUS while performing real-time imaging of an echogenic object placed in a visible lesion created at the FZ of the FUS transducer in a HIFU phantom placed in the water bath and made from a sonolucent polymer vessel filled with commercially available, optically clear HIFU phantom gel (Onda Corp). The USgFUS device can be positioned so that the FZ of the FUS transducer falls within the interior of the HIFU phantom. Subsequently operating the FUS transducer at sufficiently high intensity will raise the temperature of the HIFU phantom gel in the vicinity of the FUS FZ to over 70 degrees Celsius inducing the formation of a persistent, visibly opaque thermal lesion corresponding to the FZ of the FUS transducer. The resulting lesion which will be located at the FZ of the FUS transducer can be imaged in real time by the DIUS either directly or by placing an echogenic marker within the lesion boundary. Operating the multi-axis positioning system of the co-registration alignment system will permit the DIUS to be positioned so that the image of the lesion appears in a predetermined position within of the ultrasound image, allowing for real time visualization of the FUS FZ. When desired alignment of the DIUS and FUS FZ has been obtained, the CR positioning system can be locked in place, maintaining the co-registration of the DIUS and FUS transducer as the USgFUS treatment head is positioned for subsequent treatments.

The FUS transducer includes a structure allowing the piezoelectric crystal to be actively cooled from both the front and the back of the piezoelectric layer (for example, a 1 3 composite piezoelectric layer), which means that the FUS transducer is equipped with a thermal management module or structure. This enables the transducer to be operated at high intensity (at the piezo) for long durations and at high duty cycles up to continuous wave sonication, without thermal damage or performance drift. In addition, the FUS transducer includes thermally efficient electrically isolated lens technology allowing for heat to be removed from the therapeutic site via the transducer while it is in operation. The configuration of this structure may be, in some embodiments, similar to the one described in PCT/CA2020/051563, the content of which is incorporated herein by reference. This ensures that bubble formation at the lens face is minimized as well as reducing the water flow rate required to keep the skin cool. The transducer includes a high lateral aspect ratio aperture and is focused through a single thermally efficient lens providing a FUS beam having a compound f-number that can be optimized to provide the required focal width for single shot PNB treatment without the usual long focal length that would occur using a circular or square focal zone. In some embodiments, the transducers may produce a −3 dB focal zone of 4.5 mm laterally, 1.4 mm longitudinally, and having a focal length of ˜18 mm. This focal zone is presumed to be sufficient to treat small peripheral nerves less than 5 mm for example, in clinical applications or animal studies, using a single sonication without the need to scan or treat subsequent locations to cover the entire cross section of the PN. This approach is much more cost effective than a high element-count rectangular matrix array that could potentially also be used in such a manner. In addition, the focal zone can be created to achieve low energy in the near field with a relatively wide parameter space to optimize treatment of the target PN while simplifying the acoustic coupling and minimizing the possibility of skin burns.

Using 1.5 MHz ultrasound as an example of a typical focused ultrasound treatment frequency, the upper limit of a round spherically focused transducer is about f-number equal to 4 given typical anatomy depth relative to the skin line. Theory shows that for spherically focused transducers the −3 dB transmit focal length and the focal width are respectively equal to:

$$\begin{matrix}- 3 dB Focal length = 7.08 λ {( {F}_{number} )}^{2} \\ - 3 dB Focal width = 1.28 λ {F}_{number}\end{matrix}$$

From these equations, it can be seen that that the focal length grows as the square of the f-number compared to the focal width, which grows directly proportionally to the f-number, which presents a challenge to FUS treatment and translation from small animal targets to clinical targets for example, or from small clinical targets to larger ones, as the size of the focal zone is frequency dependent, and the lateral dimension grows much more slowly than the length direction as the f-number is increased. This leaves only the possibility of using a reasonable length focal zone that will be scanned over the required anatomy or changing the frequency of the FUS transducer to match the required anatomy. Neither of these solutions are ideal if one requires a single sonication or a limited number of sonication's within a short time at a predetermined frequency to completely treat the cross section of a nerve, with for example optimized acoustic parameters such as frequency, PRF, duty cycles or other sonication parameters that may have been found to be optimal for FUS treatment purpose, as they both require changing FUS treatment parameters between differing anatomy size or depth. Either multiple sonication's over time, or a lower frequency for larger anatomy for example could be required to achieve a single sonication of varying anatomical targets.

For example, a 4 mm wide −3 dB FZ at 1.5 MHz can generate a −3 dB focal length of somewhere between 75 mm to 100 mm deep for example when produced by a circular aperture transducer using a spherically focused lens. This focal zone could result in unintended sonication of a long cylindrical section of tissue above and below the targeted nerve for example.

In light of the above, there is a need to decouple the focal zone in at least one axis from the focal length of a single element FUS transducer.

It should be noted that as the upper limit of the focal width is approached, one limiting factor is the reduced focal gain that is achieved with high f-number lenses. In order to achieve high focal intensities, it is necessary to drive the transducer at a high transmit power level. To this end, one exemplary embodiment described herein contains a highly thermally conductive lens, for example, aluminum, in contact with an acoustically matched thermally conductive but electrically insulating ceramic composite, for example, a 1 3 composite of aluminum nitride, beryllium oxide, or alumina, for example, interposed between the piezocomposite transducer element, itself bonded to a thermally conductive and electrically conductive ground plate, for example, an aluminum plate of substantial thickness, for example ¼ lambda, ⅓ lambda, or several wavelengths thick. The 1 3 composite acoustically matched electrically isolating layer allows the lens to be actively cooled by the transducer cooling solution, while maintaining a patient current leakage level that is safe for medical devices, for example, less than 100 μA for a body floating device.

The combination of cooling of both sides of the piezo crystal and the lens of the transducer also enables very high duty cycle operation of the FUS transducer further enhancing the treatment parameter space that this device provides access to. For example, one exemplary embodiment of the technology can run at levels of pressure and intensity sufficient for treatment, continuously, while only increasing in temperature by a few degrees at the lens and without experiencing thermal performance degradation.

In some embodiments, the HARFUS transducer according to the present technique utilizes a lateral f-number of 6 and a longitudinal f-number of 1.7 to produce a 4.5 mm by 1.4 mm wide high aspect ratio −3 dB focal width with only a 1.66 cm deep focal length. This enables the treatment of anatomy relatively close to the skin in size up to 4-4.5 mm with a single sonication. In some embodiments, several of these HARFUS transducers can be collocated so that the lateral aspect of the focal zones merge into one very wide focal zone. For example, in some embodiments, three modular HARFUS transducers at 1.5 MHz can produce a −3 dB focal zone of width >10 mm at a 1.6 cm focal length.

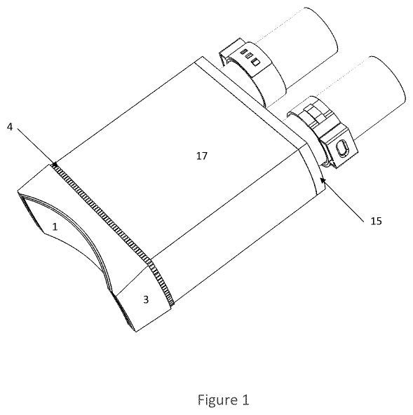

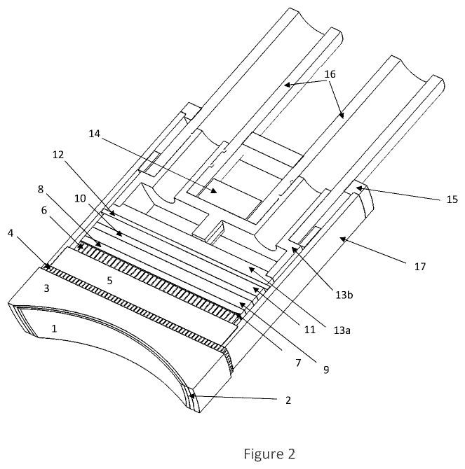

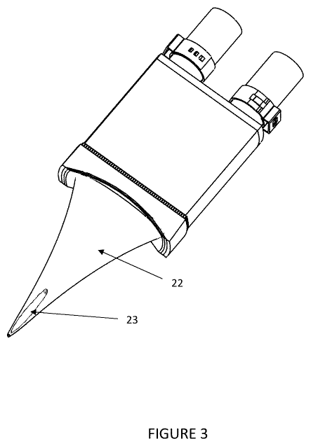

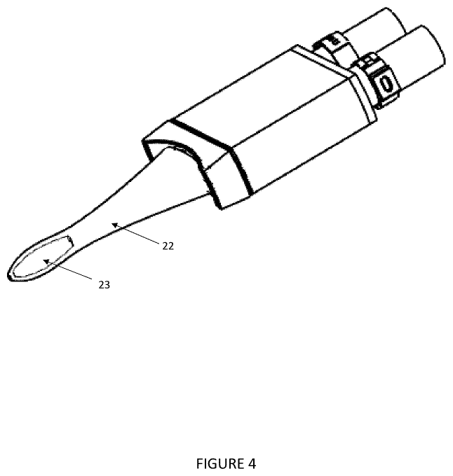

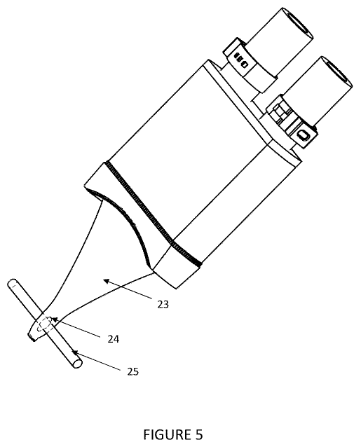

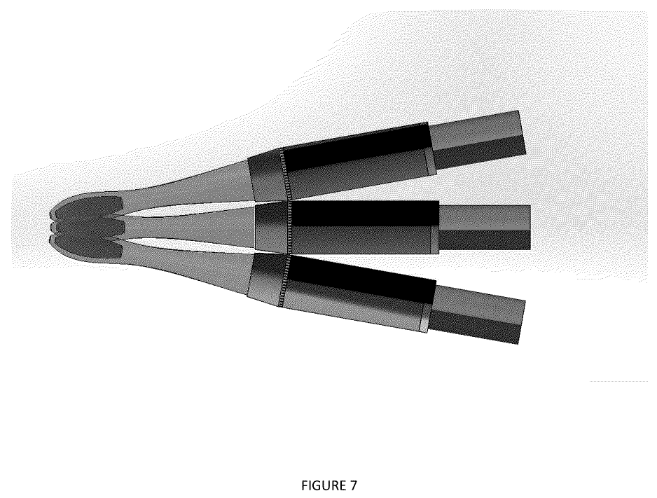

With reference to FIG. 1, there is shown an embodiment of the HARFUS transducer. The HARFUS transducer include a front matching layer 1, a rear matching layer 2, and a thermally efficient aluminum or other material lens 3. In some embodiments, the lens 3 has width and edges, which may be tapered to allow for adjacent assembly into a multi module treatment head. The HARFUS transducer also includes an acoustically matched thermally conductive composite electrically isolating layer 4 and a thermally and electrically conductive layer—also acts as redundant ground 5. Still referring to FIG. 1, the HARFUS transducer includes a high aspect ratio rectangular aperture material specific single element piezo composite 6, matched to aluminum (at about 17 MR). Of note, the apertures can be elliptical aperture or other high aspect ratio shape to optimize acoustic field as required. The HARFUS transducer also includes a de-matching layer 7, with preferably high acoustic impedance. The de-matching layer 7 is preferably thermally and electrically conductive, may be made from material such as tungsten, tungsten carbide or other molybdenum for example. The HARFUS transducer also includes a dual layer dematching backing (DLDB) ¼ lambda layer 8 (with low acoustic impedance, the layer 8 being made from an electrically and thermally conductive material, such as graphite), such as the one described in PCT/CA2020/051563. The HARFUS transducer also includes a DLDB ¼ lambda layer 9 (with high acoustic impedance layer, the layer 9 being made from an electrically and thermally conductive material, such as tungsten). The HARFUS transducer also includes a DLDB ¼ lambda layer 10 (with low acoustic impedance, the layer 10 being made from an electrically and thermally conductive material, such as graphite). The HARFUS transducer also includes a DLDB layer 11 (with high acoustic impedance layer, the layer 11 being made from an electrically and thermally conductive, such as copper, tungsten, or any other conductive high acoustic impedance material(s)). The HARFUS transducer also includes an aluminum nitride layer 12. The layer 12 may be replaced by a layer made from any other thermally conductive materials that is also electrically insulating such as boron nitride, beryllium oxide, aluminum oxide or other suitable thermally conductive and electrically insulating materials. The HARFUS transducer also includes a heat sink, which may be embodied by liquid cooling block base 13A and liquid cooling block body 13B for the liquid cooling block 13A, or a finned air heat sink including a plurality of fins. In some embodiments, air may be used as a cooling mechanism. In some embodiments, the cooling water block may be made from a single component, or many components. The HARFUS transducer also includes an aluminum nitride layer 14. The layer 14 may be replaced by a layer made from any other thermally conductive electrically insulating materials that can form the thermal return path plate to couple the thermal energy from the front (5) plate to the back of the liquid cooling block. The HARFUS transducer also includes an electrical shield 15, which may be embodied by a copper housing lid. The shield 15 is made from an electrically and thermally conductive material, which allow it to form an electrical shield and possibly ground connection path for RF connector (not shown). The HARFUS transducer also includes liquid cooling inlet/outlet tubes 16. The HARFUS transducer also includes an electrically and thermally conductive housing 17, which forms both the electrical ground connection to the transducer and the thermal cooling path for the front face of the piezocomposite. Other visual representations of the HARFUS transducer are illustrated in FIGS. 2 to 7.

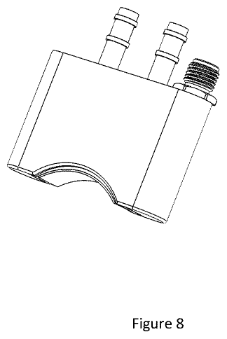

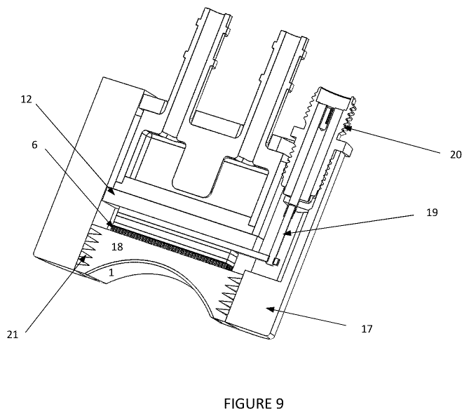

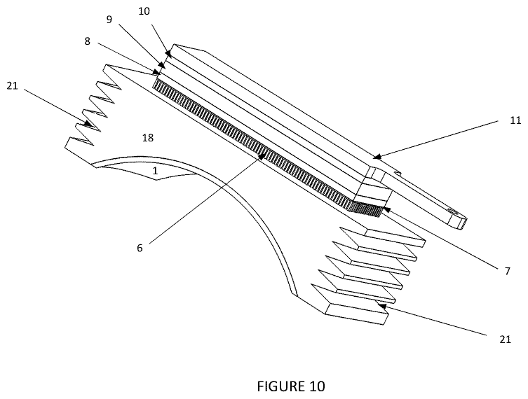

Now turning to FIGS. 8 to 10, a modular HARFUS transducer will be described. In these Figures, there is illustrated a 3-module treatment FUS system having co-aligned focal zones positioned to allow the −3 dB lateral focal zones of each module to merge in one axis. The lateral merged focal zone has a −3 dB focal width of approximately 3 times a single module. Of note, the three focal zones maintain approximately the same focal width in the orthogonal direction to the lateral field and maintain approximately the same −3 dB focal length as a singe module. The result is a wide focal zone that is many times wider and many times shorter than what can be produced by a single element FUS transducer at a given frequency.

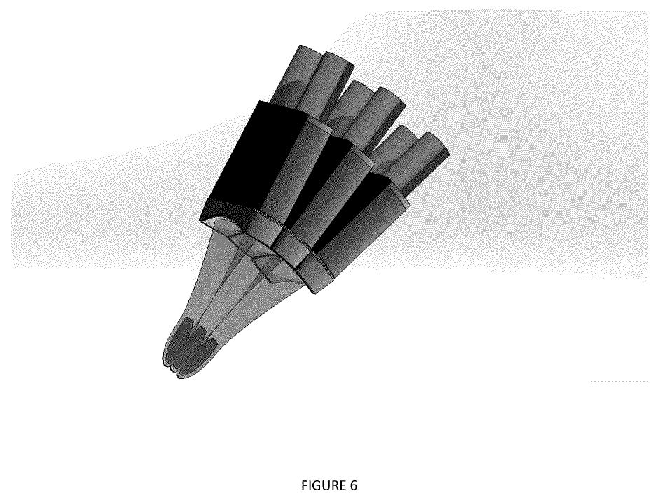

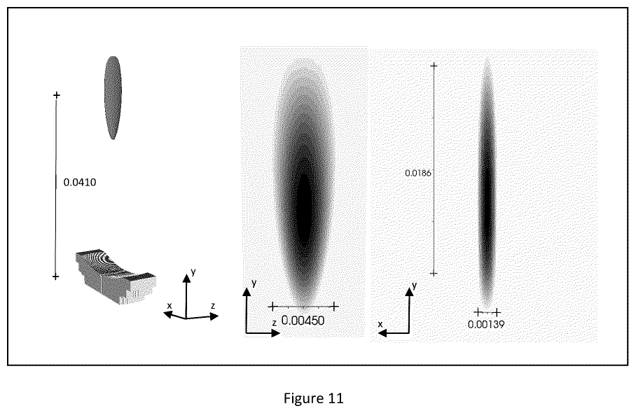

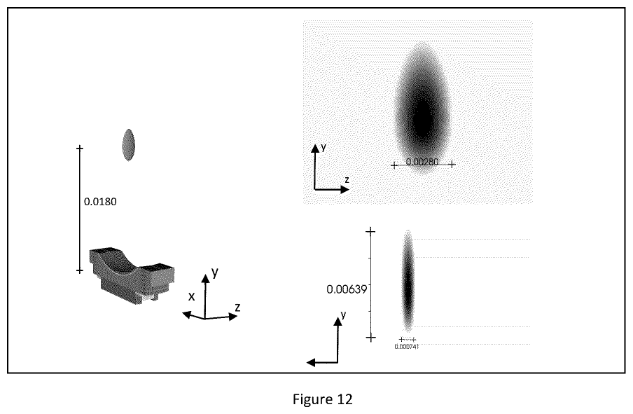

Now turning to FIGS. 11 and 12, results that can be obtained with the present techniques are illustrated (lateral results, −3 dB). FIG. 11 shows a lateral focal zone of the high aspect ratio FUS transducer having a focal zone of 4.5 mm wide by 1.4 mm wide and only 1.66 cm long, occurring at a depth of approximately 3.5 cm-4 cm from the skin line. The HARFUS transducer enables direct translation of research on single sonication neuromodulation (NM) of a peripheral nerve (PN) at for example 1.5 MHz, on subjects ranging from, for example, a rat to a larger animal model, and through to clinical studies using the same frequency and sonication parameters. Translational studies can be done, from small animal through to clinical studies, at a single frequency, for example at 1.5 MHz, and using a single set of FUS parameters. This can be accomplished using several of the exemplary HARFUS transducers disclosed herein, such as those shown in FIGS. 8, followed by that shown in FIG. 1, and then onto that shown in FIG. 6. This sequence of multiple HARFUS transducers, each operating at 1.5 MHz for example, one optimized for small animal based HARFUS treatment producing a relatively small focal zone of about 2.5 mm by <1 mm by about 6.7 mm long, and one larger 1.5 MHz HARFUS treatment head producing a larger width focal zone, for example 4.5 mm by 1.3 mm by about 17 mm, and finally the use of a a multiple modular treatment head containing several 1.5 MHz HARFUS transducers co-located to laterally merge focal zones for up to 10.6 mm by 1.3 mm wide by 1.66 cm long −3 dB FZ. It is of course understood that the present technology can be applied over a wide range of frequencies to many possible FUS applications in addition to neuromodulation. One key aspect of the present technology being the possibility to scale the lateral width and axial length of the focal zone linearly over a wide parameter space at a given frequency thus enabling translational research between small animals, as well as freeing the use of optimal acoustic parameters to a wide variety of anatomical structures at varied depths below the skin line.

It should be noted that to obtain a 3.5 mm wide acoustic focal zone at 1.5 MHz using a single element FUS transducer according to the prior art method, for example having a round aperture, one will produce a focal length of about 80 mm long (−3 dB). occurring at approximately 7.5 cm from the skin line. This result can be readily simulated using a round single element FUS model having approximately an f-number of 4.5 nominal aperture but a short focal length requiring a relatively small k wavenumber aperture product of only 65, resulting in a diffraction limited focal width of less than the f-number. Note that to achieve a wider focal zone with this type of transducer from prior art, a much larger transducer aperture would be required, with a focal zone moving much deeper.

While it may also be possible to use diffraction correcting lenses beyond what is used in the present techniques, it is not likely to significantly overcome the exponential growth of the focal length vs focal width vs increasing f-number. Such a prior art device could not safely be used to treat small targets such as a nerve, especially near the skin or above or below sensitive organs or structures, having an 8 cm focal length.

Using finite element modelling techniques, traditional round single element FUS transducer geometries can be shown to have an upper −3 dB focal zone width limitation approximately 4 mm at 1.5 MHz occurring with a nearly unusable focal length of over 80 mm. These numbers agree with theory which predicts the lateral focal zone to be proportional to f-number times wavelength, and the focal length to be proportional to a constant (about 7 for a typical single element FUS transducer) time the f-number squared times lambda. This is a key reason that a focal zone of 4-5 mm required to sonicate for example, even a relatively small 4 mm diameter peripheral nerve, is such a challenge. One skilled in the art will understand that much neuromodulation research ahs be done at 1.5 MHz, and will further know that lambda is approximately 1 mm at 1.5 MHz in tissue, meaning an f=number of 4-5 is required to achieve the focal zone width that can sonicate a 4 mm diameter nerve in one sonication, and also meaning that the focal zone will be about 7 times 4 to 5 times the size of the lateral focal zone, or about 14 cm long when the focal zone is 4.5 mm long.

Using the present HARFUS transducer allows the production of a much shorter 16.6 mm long focal length while producing a 4.5 mm wide by 1.4 mm thick focal zone.

One exemplary embodiment of the technology is a small animal ultrasound guided Focused Ultrasound (USgFUS) device including a liquid cooled FUS transducer contained in an articulating treatment head further including a micro-positioning and clamping mechanism providing for the co-registration of the imaging plane of a suitable high-resolution US diagnostic imaging ultrasound scanner (DIUS) with the focal zone (FZ) of the FUS transducer. Additionally, the device includes a water bath and small animal handling platform with a positioning system for orienting the co-registered FUS and imaging transducers with respect to the rat enabling the precise visualization of PN and subsequent insonation to achieve an accurate PNB.

In this exemplary embodiment the liquid cooled FUS transducer includes an acoustic stack incorporating material specific piezo composite material specific stack, and may or may not include a composite dielectric isolation layer between the material specific composite piezo, an aluminum ground reenforcing electrode and cooling layer, and the aluminum acoustic lens, allowing highly thermally efficient operation and making it possible to perform FUS treatments at up to 100% duty cycle for long durations with almost no heating of the device or lens face, enabling a very wide parameter space to be tested. Nonlimitative examples of the stacks, structures and materials are presented in PCT/CA2019/051046, PCT/CA2020/051563 and/or PCT/CA2022/050387.

Furthermore, the FUS transducer includes high lateral aspect ratio aperture and lens technology, having an elevation aperture (or short axis aperture) of 4 mm and an azimuthal aperture (or long axis aperture) of 18 mm and a focal depth of 15 mm resulting in a relatively shorter focal length while providing the required focal width to treat for example an entire peripheral nerve in a small animal model, for example a rat, in one sonication. In one embodiment, intended for small animal experiments, the transducer provides a focal spot having a nominal −3 dB azimuthal focal width of approximately 2.8 mm, an elevation focal width of <1.0 mm and a nominal −3 dB focal length of approximately 6.8 mm, with the center of the focal spot occurring approximately 15 mm from the face of the transducer. These FZ parameters are expected to facilitate for example, accurate neuromodulation of a peripheral PN in a small animal model such as a rata, with one sonication while minimizing the possibility of skin burns. Co-registration (CR) of the high-resolution DIUS to the FZ of the FUS transducer is accomplished by positioning the DIUS while performing real-time imaging of an echogenic object placed in a visible lesion created at the FZ of the FUS transducer in a HIFU phantom placed in the water bath and made from a sonolucent polymer vessel filled with commercially available, optically clear HIFU phantom gel (Onda Corp). USgFUS device can be positioned so that the FZ of the FUS transducer falls within the interior of the HIFU phantom. Subsequently operating the FUS transducer at sufficiently high intensity can raise the temperature of the HIFU phantom gel in the vicinity of the FUS FZ to over 70 degrees Celsius inducing the formation of a persistent, visibly opaque thermal lesion corresponding to the FZ of the FUS transducer. The resulting lesion which can be located at the FZ of the FUS transducer can be imaged in real time by the DIUS either directly or by placing an echogenic marker within the lesion boundary. Operating the multi-axis positioning system of the co-registration alignment system permits the DIUS to be positioned so that the image of the lesion appears in a predetermined position within of the ultrasound image, allowing for real time visualization of the FUS FZ. When desired alignment of the DIUS and FUS FZ has been obtained, the co-registration (CR) positioning system can be locked in place, maintaining the co-registration of the DIUS and FUS transducer as the USgFUS treatment head is positioned for subsequent treatments.

In another embodiment intended to provide single location single sonication neuromodulation in a small peripheral nerve of approximately 4 mm, the USgFUS transducer includes an elevation aperture of about 7 mm and azimuthal aperture of about 24 mm with a focal depth occurring at approximately 3.5 cm. The investigational ultrasound guided FUS device can be used to, for example, perform transcutaneous PNB in a peripheral nerve up to 4 mm in diameter from a single position and using a single sonication. The device includes a custom 1.5 MHz liquid cooled high lateral aspect ratio aperture FUS transducer providing low aspect ratio FZ for the safe single shot treatment of PN located, for example from 2 cm to 5 cm below the skin. In addition, the device includes a multi-jointed locking articulating arm or robotic arm to facilitate the placement of the FUX FZ with respect to the anatomy to be treated. Guidance to the treatment location can be provided by a co-registered high resolution diagnostic imaging array (DIA) such as a Sonosite L38 10-5 linear array for example, and diagnostic Ultrasound (DUS) system. A means of co-registering the DIUS and FUS FZ includes into the device, including facility for placing a HIFU compatible phantom filled with, for example, thermally sensitive HIFU phantom gel (Onda Corp) to enable visualization of the focal zone of the FUS transducer. Once co-registration is complete, the treatment head, comprising both the FUS transducer, the DIUS co-registration mechanism, and the DIUS, as well as a means of acoustically coupling the US transducer to the subject can be positioned by means of the articulating arm, using the DIUS and US imaging system to provide real time guidance to the PN to be treated.

When an optimal position and acoustic treatment window is reached, the articulating arm can be locked in place to allow for precise treatment over the duration of the FUS sonication.

The FUS transducer includes structure allowing the piezoelectric crystal to be actively cooled from both the front and the back of the piezo. This enables the transducer to be operated at high intensity (at the piezo) for long durations and at high duty cycles up to continuous wave sonication, without thermal damage or performance drift. In addition, the transducer includes thermally efficient electrically isolated lens technology allowing for heat to be removed from the therapeutic site via the transducer while it is in operation. This ensures that bubble formation at the lens face is minimized as well as reducing the water flow rate required to keep the skin cool. The design includes a high lateral aspect ratio aperture and is focused through a single thermally efficient lens providing a FUS beam having a compound f-number that can be optimized to provide the required focal width for single shot FUS treatment without the usual long focal length that would occur using a circular or square focal zone. One exemplary embodiment comprises a single FUS transducer that produces a −3 dB focal zone of 4.5 mm laterally, 1.3 mm longitudinally, and having a −3 dB focal length of −17 mm. This focal zone is presumed to be sufficient to treat a nerve of 4 mm or less lying at least 1.5 cm below the skin line without the need to scan the FZ or treat subsequent locations to cover the entire cross section of the PN. This approach is much more cost effective than a high element-count rectangular matrix array that could potentially also be used in such a manner. In addition, the focal zone can be created to achieve low energy in the near field with a relatively wide parameter space to optimize treatment of the target PN while simplifying the acoustic coupling and minimizing the possibility of skin burns.

Ultrasound image guidance of the FUS treatment is critical to achieving an accurate transcutaneous sonication of target anatomy, for example a peripheral nerve. It is important to note that FUS sonication cannot be directly visualized in vivo during treatment without the use of contrast enhancement agents, and in some cases elastography or ARFI techniques or MRI methods, however, all of these options are costly, and some limits due to the lower levels of insonation associated with NM FUS can render these techniques ineffective. In one exemplary embodiment, the FUS transducer is mounted within a system which includes a means of co-registering a high-resolution DIUS with the focal zone of the FUS transducer by means of a sonolucent hollow cylindrical tissue mimicking phantom, having an outside diameter of approximately 5 cm and an inner diameter of approximately 4 cm and an absorbing posterior wall. The phantom will be filled with a clear HIFU phantom gel (ONDA Corp) that changes to a stable opaque material when the temperature of the gel is raised by thermal FUS ablation within the gel. The treatment head of the FUS transducer will be positioned over the custom HIFU phantom, and both the FUS transducer and DIUS transducer will be acoustically coupled to the phantom using the same method of acoustic coupling as will be used for treatment of, for example a peripheral nerve. The FUS transducer will then be driven at suitable levels to promptly induce a thermal lesion in the phantom that will correspond to the FZ of the FUS transducer. The lesion will then be visualized by the DIUS and ultrasound imaging system, with the provision to adjust the relative position of the DIUS within the treatment head to achieve centering of the lesion on the screen of the ultrasound imaging system.

When relative positioning is achieved as desired, for example, centering of the longitudinal FZ on the DUS image, the relative position of the DIUS will be locked, leaving the operator free to position the treatment head on the subject using the co-registered DUS image to find the optimal treatment location for the FUS treatment. When the optimal location has been obtained, the articulation arm can be locked by means of a remote switch such as a foot switch or button on the treatment head. Treatment can then proceed.

In accordance with one aspect, there is provided a method for aligning a treatment head including a HARFUS transducer and a diagnostic ultrasound imaging system with a sample to characterize, the method including acoustically coupling the treatment head with an ultrasound-sensitive phantom; performing a co-registration sonication sequence with the HARFUS transducer to determine an acoustic focal zone using the ultrasound-sensitive phantom; positioning an imaging plane of the treatment head in a center of the acoustic focal zone of the HARFUS transducer; locking a position of the HARFUS transducer with respect to the treatment head; acoustically coupling the treatment head with respect to the sample to characterize, based on the locked positioned of the HARFUS transducer; and operating the treatment head to characterize the sample.

The initial design of the FUS transducer has been simulated using OnScale numerical simulation software using models that have been validated at over 30 W/cm² intensity at the transducer element. The focal zone of the proposed transducer can be achieved using a high aspect ratio aperture having dimensions of approximately 6 mm by 20 mm, and a focal depth of approximately 3.5 cm for a hybrid F-number of 6/1.8. According to simulation results, the initial design provides a focal zone having a longitudinal −3 dB focal width of 1.3 mm (relative to the peripheral nerve) and a transverse −3 dB focal width of 4.5 mm. Simulations of the −3 dB focal zone are shown below in figure

The focal depth and FZ size can be tailored during development by changing the size and aspect ratio of the aperture as well as the focal characteristics of the lens. The FUS device enables sub millimeter positional accuracy of the treatment head, and sub-millimeter co-registration with a high frequency DIUS and DIS to enable FUS treatments such as neuromodulation, to be carried out with a high degree of spatial accuracy, and in a short space of time with minimal training. In addition, when paired with a suitable RF power source of 150 W for example, the FUS transducer should be capable of producing peak intensities of over 1000 W/cm² continuously for minutes over a focal zone of 4.5 mm by 16 mm.

Of note, the present technology, or at least some of the embodiments thereof, is compatible with the technologies described in PCT/CA2019/051046, PCT/CA2020/051563 and PCT/CA2022/050387, the content of which is herein incorporated by reference.

Now turning to the advantages and benefits of the present techniques, the need to partially decouple the frequency of focused ultrasound treatment from the size of the focal zone in a cost-effective single element solution is significant. The further ability to combine such devices in a modular fashion to expand the focal zone laterally without increased risk of skin burns, or the need to add costly 2D arrays and the associated electronics and treatment planning software and image guidance is real and is holding back the adoption of FUS in the clinic.

Furthermore, the need to translate preclinical work with the same parameters on small animals to clinical studies having much larger anatomical structures, but similar tissues is a significant barrier to the translation of preclinical FUS results to the clinic. The ability of the HARFUS device disclosed herein to produce identical acoustic properties in a focal zone that can change by a full order of magnitude is potentially groundbreaking advance for the translation of preclinical FUS studies to the clinic.

All of this is enhanced by the possibility to use diagnostic ultrasound to guild these HARFUS devices to the target anatomy. The safety and practicality of US guided FUS (USgFUS) is enhanced dramatically by the ability to manage the focal length of the target while matching the width of the focal zone to the target anatomy, meaning complex treatment planning of a small focal zone within a larger anatomy, often requiring complex software and MRI guidance, and long treatment times, is not required.

The present techniques present high aspect ratio aperture which produces a wide lateral focal zone with a relatively short focal length combined with exceptional cooling of both sides of the piezo element and the lens enables both FUS research and clinical treatment at a lower cost and with greater efficacy where a single sonication of the anatomy is significant and can reduce treatment time and cost over complex 2D array based treatments.

Several alternative embodiments and examples have been described and illustrated herein. The embodiments described above are intended to be exemplary only. A person skilled in the art would appreciate the features of the individual embodiments, and the possible combinations and variations of the components. A person skilled in the art would further appreciate that any of the embodiments could be provided in any combination with the other embodiments disclosed herein. The present examples and embodiments, therefore, are to be considered in all respects as illustrative and non-restrictive. Accordingly, while specific embodiments have been illustrated and described, numerous modifications come to mind without significantly departing from the scope defined in the current description and appended claims.

1. A high aspect ratio aperture focused ultrasound (HARFUS) transducer, the HARFUS transducer comprising:
a high aspect ratio acoustic stack having a front end and a rear end, the acoustic stack comprising:
a composite piezoelectric layer, the composite piezoelectric comprising a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture;
a thermally and electrically conductive layer in physical contact with the piezoelectric layer, the composite piezoelectric layer being acoustically matched with the thermally and electrically conductive layer;
a thermally conductive and electrically isolating layer in contact with the thermally and electrically conductive layer;
a lens in contact with the thermally conductive and electrically isolating layer; and
at least one matching layer at least partially extending over the lens, wherein the acoustic stack is configured to produce a field defining an acoustic focal zone, the acoustic focal zone having a relatively wide lateral dimension, a relatively narrow lateral dimension, and a relatively low focal length to focal width ratio; and
a backing structure contacting the rear end of the acoustic stack.

2. The HARFUS transducer of claim 1, wherein the acoustic focal zone is shaped like a pumpkin seed.

3. The HARFUS transducer of claim 1, wherein said at least one matching layer comprises a front matching layer extending over a rear matching layer.

4. The HARFUS transducer of claim 1, wherein the lens has tapered edges and a tapered width.

5. The HARFUS transducer of claim 1, wherein the lens has an outer periphery, the outer periphery being threaded or including anechoic features.

6. The HARFUS transducer of claim 1, wherein the lens curvature is a spherical curvature or an elliptical curvature.

7. The HARFUS transducer of claim 1, further comprising a de-matching layer contacting the rear end of the high aspect ratio acoustic stack, the de-matching layer directly contacting one of: the composite piezoelectric layer and the backing structure.

8. The HARFUS transducer of claim 1, wherein the backing structure is a dual layer de-matching backing structure.

9. The HARFUS transducer of claim 1, further comprising a heat sink.

10. A modular system, the modular system comprising a plurality of HARFUS transducers as claimed in claim 1, arranged to result in a laterally merged focal zone having a lower axial focal length to lateral focal width ratio than a single HARFUS transducer.

11. A high aspect ratio aperture focused ultrasound (HARFUS) transducer, the HARFUS transducer comprising:
a high aspect ratio acoustic stack having a front end and a rear end, the acoustic stack comprising:
a composite piezoelectric layer, the composite piezoelectric comprising a plurality of regions made of a piezoelectric material, each region being separated one from another by a non piezoelectric matrix material, the composite piezoelectric layer having a length and a width defining a high aspect ratio, wherein the length and width of the composite piezoelectric layer form a high aspect ratio acoustic aperture;
a thermally and electrically conductive layer in physical contact with the piezoelectric layer, the composite piezoelectric layer being acoustically matched with the thermally and electrically conductive layer;
a lens in contact with the thermally and electrically conductive layer;
at least one matching layer at least partially extending over the lens, wherein the acoustic stack is configured to produce a field defining an acoustic focal zone, the acoustic focal zone having a relatively wide lateral dimension, a relatively narrow lateral dimension, and a relatively low focal length to focal width ratio; and
a backing structure contacting the rear end of the acoustic stack.

12. The HARFUS transducer of claim 11, wherein the acoustic focal zone is shaped like a pumpkin seed.

13. The HARFUS transducer of claim 11, wherein said at least one matching layer comprises a front matching layer extending over a rear matching layer.

14. The HARFUS transducer of claim 11, wherein the lens has tapered edges and a tapered width.

15. The HARFUS transducer of claim 11, wherein the lens has an outer periphery, the outer periphery being threaded or including anechoic features.

16. The HARFUS transducer of claim 11, wherein the lens curvature is a spherical curvature or an elliptical curvature.

17. The HARFUS transducer of claim 11, further comprising a de-matching layer contacting the rear end of the high aspect ratio acoustic stack, the de-matching layer directly contacting one of: the composite piezoelectric layer and the backing structure.

18. The HARFUS transducer of claim 11, wherein the backing structure is a dual layer de-matching backing structure.

19. The HARFUS transducer of claim 11, further comprising a heat sink.

20. A modular system, the modular system comprising a plurality of HARFUS transducers as claimed in claim 11, arranged to result in a laterally merged focal zone having a lower axial focal length to lateral focal width ratio than a single HARFUS transducer.

21. A method for aligning a treatment head comprising a HARFUS transducer and a diagnostic imaging system with a sample to characterize, the method comprising:
acoustically coupling the treatment head with an ultrasound-sensitive phantom;
performing a co-registration sonication sequence with the HARFUS transducer to determine an acoustic focal zone using the ultrasound-sensitive phantom;
positioning an imaging plane of the treatment in a center of the acoustic focal zone of the HARFUS transducer;
locking a position of the HARFUS transducer with respect to the treatment head;
acoustically coupling the treatment head with respect to the sample to characterize, based on the locked positioned of the HARFUS transducer; and
operating the treatment head to characterize the sample.
